(19) United States
(12) Plant Patent Application Publication
(10) Pub. No.: US 2012/0304347 P1
(43) Pub. Date: Nov. 29, 2012

(54) Osteospermum plant named 'SAKOST8202'

(50) ;

(75) Inventor: Niels G. Kristensen, Silkeborg (DK)

(73) Assignee: Sakata Seed Corporation, Yokohama (JP)

(21) Appl. No.: 13/134,031

(22) Filed: May 26, 2011

(51) Int. Cl. A01H 5/00

(52) U.S. Cl. PLT/360

(57) ABSTRACT

A new Osteospermum plant named ‘SAKOST8202’ particularly distinguished by having light pink colored flowers and a semi-erect plant habit, is disclosed.

### GENUS AND SPECIES

Osteospermum hybrida

### VARIETY DENOMINATION

‘SAKOST8202’

### BACKGROUND OF THE NEW PLANT

The present invention comprises a new and distinct cultivar of Osteospermum hybrida referred to by the variety name ‘SAKOST8202’. ‘SAKOST8202’ originated from a hybridization of hybrid Osteospermum proprietary breeding lines ‘205011’ and ‘205104’ at Petersminde Nursery in Aabyhoej, Denmark. The female parent, ‘205011’ has a light pink flower color with a grey center and a compact growth habit. The male parent, ‘205104’, has a light pink flower color with a grey center and a semi-erect plant habit.

In May 2006, the two Osteospermum lines were crossed and seeds were obtained. The seeds were sown and 200 plants were grown in pots for evaluation. Out of 200 F₁ lines, the breeder selected plant number 82, which has light pink colored flowers (young flowers have a whitish flower color) and a semi-erect plant habit.

In February 2007, plant number 82 was vegetatively propagated by cuttings and re-evaluated in an open field and a greenhouse. Plant number 82 was given the breeder code number ‘207106’. The breeder confirmed the stability of the distinct characteristics of this variety.

In spring 2007, plants were evaluated again in pots and in an open field. The selection was named ‘SAKOST8202’ and was found to reproduce true to type in successive generations of asexual propagation.

### SUMMARY OF THE INVENTION

The following are the most outstanding and distinguishing characteristics of the new variety when grown under normal horticultural practices in a greenhouse in Salinas, Calif.

1. Light pink flowers; and

2. Semi-erect plant habit.

### DESCRIPTION OF THE PHOTOGRAPHS

The new Osteospermum plant is illustrated by the accompanying photographs which show the overall plant habit including form, foliage, and flowers. The photographs are of a plant grown four months from transplant date and five months from stick date into 4-inch pots from rooted cuttings in Salinas, Calif. under greenhouse conditions. The colors shown are as true as can be reasonably obtained by conventional photographic procedures.

FIG. 1 shows overall plant habit of the plant grown in a pot.

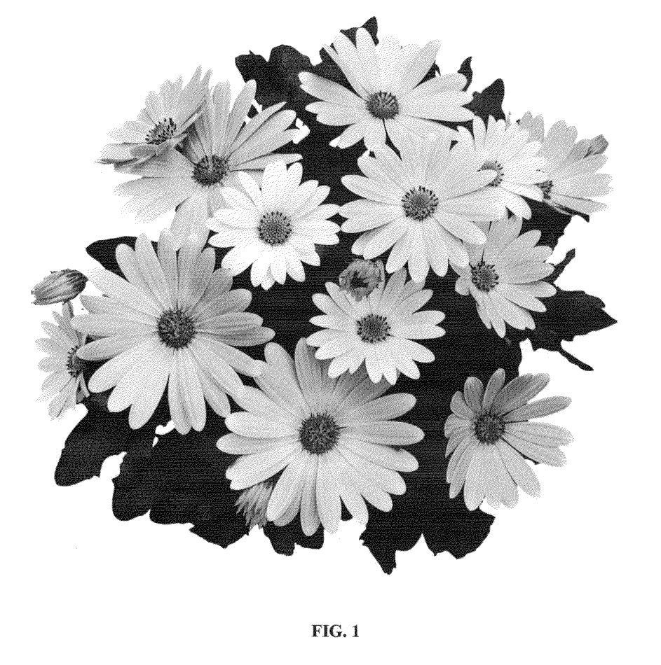

FIG. 2 shows the mature inflorescence of the plant.

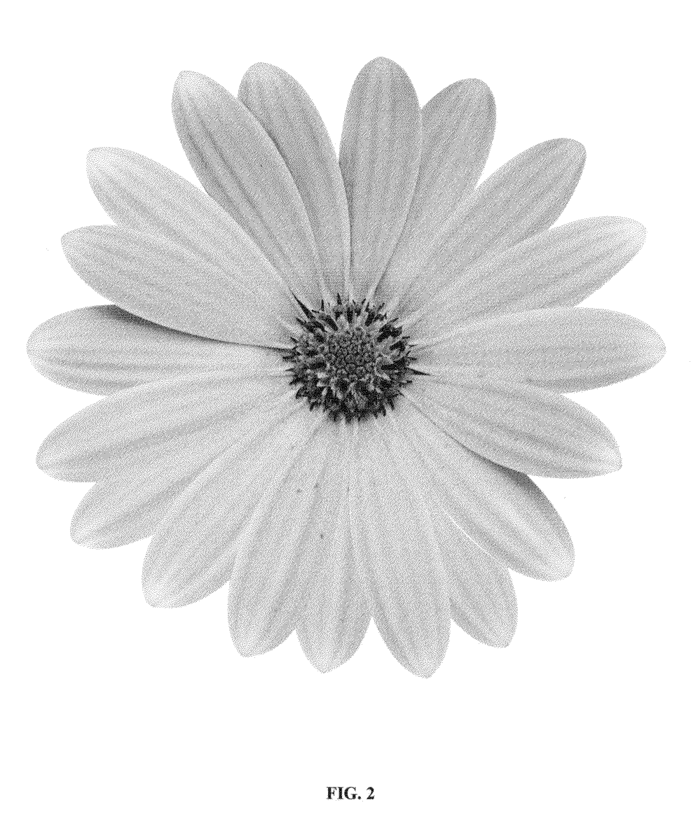

FIG. 3 shows three flowers with leaves.

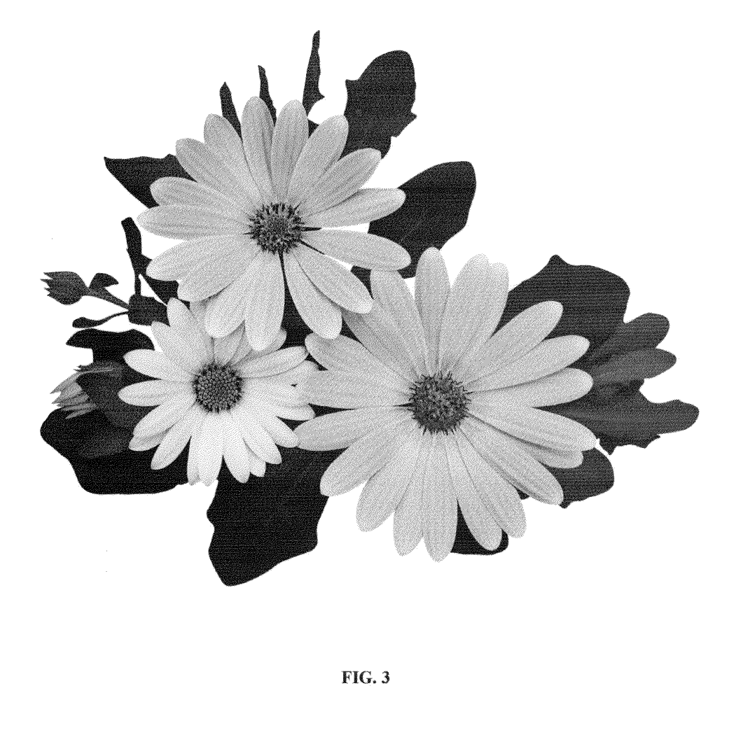

### DESCRIPTION OF THE NEW VARIETY

The following detailed descriptions set forth the distinctive characteristics of ‘SAKOST8202’. The data which define these characteristics were collected from asexual reproductions carried out in Salinas, Calif. Data were collected on plants grown approximately four months from transplant date and five months from stick date into 4-inch pots under greenhouse conditions in Salinas, Calif. Color references are to the R.H.S. Colour Chart of The Royal Horticultural Society of London (R.H.S.), 4^{th} edition.

### DESCRIPTION OF THE NEW PLANT

Classification:
   Family.—Compositae.
   Botanical.—Osteospermum hybrida.
   Common.—Osteospermum, Cape Daisy Softly Pink.
Parentage:
   Female parent.—Proprietary Osteospermum line ‘205011’.
   Male parent.—Proprietary Osteospermum line ‘205104’.
Growth:
   Time to produce a rooted cutting.—Cuttings will colonize a 2.5 cm diameter by 2.5 cm tall greenhouse tray cell with peat-based plant media in approximately four weeks. Cuttings are dipped in a normal dilution (1:9) of Dip 'N Grow root inducing solution in water. The trays are misted hourly during rooting.
   Environmental conditions for plant growth.—Rooted cuttings are transplanted to pots with a 16 cm. diameter, one plant per pot. Peat-based growing media is used. The pots are watered using a 150-200 ppm fertilizer solution using 18-8-18 fertilizer. The soil is allowed to dry between watering. During the first few weeks after transplanting the plants should have evening temperatures around 15°-18° C. for good root growth. When plants reach 7.5-10 cm in height they are pinched back to 5-6 leaves to promote branching. Spring and summer daytime high temperatures in Salinas, Calif., where the data was collected, range from 16°-25° C.
   Time to bloom from propagation.—Approximately four weeks when rooted vegetative cuttings are transferred to a 5 inch diameter pot. Flowering season is all year in the United States. Vernalization is not required to induce flowering.
Plant description:
   Habit.—Semi-erect.
   Life cycle.—Perennial.
   Form.—Semi-erect.
   Height (from soil line to first node).—1.0 cm.
   Height (from soil line to top of foliage).—35.0 cm to 36.0 cm.
   Width.—38.0 cm to 40.0 cm.
Stems:
   General.—Multiple.
   Stem length (total).—10.0 cm to 12.0 cm.
   Diameter.—0.2 cm.
   Internode length.—1.5 cm.
   Color.—Green RHS 144A (Yellow-green).
   Anthocyanin color.—Slight, RHS 187A (Greyed-purple).
   Pubescence.—Slight Color: RHS N155A (White) Shape: Short, hair-like fibers.
Branches:
   General.—Circular cross-section.
   Number of main branches.—1.
   Total number of branches.—8.
   Length.—13.0 cm to 14.0 cm, approximately 1.0 cm from soil line to first node, 1.0 cm between first and second nodes.
   Diameter.—0.4 cm.
   Internode length.—2.5 cm.
   Color.—RHS 144A (Yellow-green).
   Anthocyanin color.—Slight, RHS 187A (Greyed-purple).
   Pubescence.—Glabrous.
Leaves:
   Arrangement.—Alternate.
   Shape.—Oblanceolate.
   Apex.—Acute.
   Base.—Attenuate.
   Margin.—Serrate.
   Venation.—Pinnate.
   Surface.—Dull.
   Surface pubescence.—Very slight, mainly around edge.
   Pubescence color.—RHS N155A (White).
   Attachment.—Decurrant.
   Length.—7.5 cm.
   Width.—3.0 cm.
   Thickness.—Less than 0.1 cm.
   Color.—Upper surface: RHS 137A (Green) Lower surface: RHS 137B (Green).
   Venation color.—Upper surface: RHS 144B (Yellow-green) Lower surface: RHS 144C (Yellow-green).
Inflorescence:
   Number per plant.—13.
   Type.—Capitulum (head); disk florets are perfect and ray florets are carpellate.
   Flowering habit.—Determinate.
   Lastingness of flowers.—3 to 4 days.
   Fragrance.—Absent.
   Seed production.—Absent.
   Diameter.—7.0 cm.
   Depth.—1.0 cm.
   Petal number.—18 to 22 per inflorescence.
   Disc diameter.—1.2 cm.
   Disc floret number.—60 to 80 per inflorescence.
   Peduncle length.—6.0 cm from inflorescence to first node.
   Peduncle diameter.—0.2 cm.
   Peduncle color.—RHS 144A (Yellow-green).
   Peduncle texture.—Dull, slight pubescence.
   Phyllaries.—Arrangement: 15 to 20 per inflorescence, free, arranged symmetrically Length: 1.1 cm Width: 0.1 cm Apex: Acute Margin: Entire Shape: Linear, acute Color: Both surfaces: RHS 137A (Yellow-green).
Ray florets (ligules):
   Corolla.—1 petal per ray floret; only the outer row of florets are the ray florets.
   Number of ray florets per inflorescence.—18 to 22.
   Petal length.—3.5 cm.
   Petal width.—1.0 cm.
   Petal shape.—Lanceolate.
   Petal apex.—Acute.
   Petal margin.—Entire.
   Petal color.—Upper surface: RHS 62C (Red-purple) with young flowers of RHS 155B (White) Lower surface: RHS 151B (Yellow-green).
   Petal pubescence.—Glabrous.
Disc florets:
   Bud color.—RHS N187A (Greyed-purple).
   Shape.—Tubular.
   Apex.—Pointed.
   Surface.—Dull, pubescent.
   Size.—Length: 0.6 cm Width: 0.1 cm.
Reproductive organs:
   Ovary.—Superior.
   Pistil form.—1 style with 2 stigma branches.
   Pistil length.—0.5 cm.
   Stigma color.—RHS N187A (Greyed-purple).
   Style color.—RHS 155A (White).
   Stamens.—5 fused into a single tube.
   Anther color.—RHS N187A (Greyed-purple).
   Pollen color.—RHS 17A (Yellow-orange).
   Filament color.—RHS 155A (White).
Disease and insect resistance: ‘SAKOST8202’ is very disease resistant; the plants are susceptible to aphids, thrips, whiteflys and worms.

### COMPARISON WITH PARENTAL LINES AND KNOWN VARIETIES

‘SAKOST8202’ is a distinct variety of Osteospermum owing to its light pink colored flowers and a semi-erect plant habit. ‘SAKOST8202’ is distinguished from its parents as described in Table 1. Color references are to The Royal Horticultural Society Colour Chart, 4^{th} Edition.

TABLE 1
Comparison with Parental Lines

| | | Male Parent | Female Parent |
|---|---|---|---|
| Characteristic | ‘SAKOST8202’ | ‘205104’ | ‘205011’ |
| Ray floret (ligule) | RHS 62C (Red-Purple) | Light pink | Light pink |
| color, upper | with young flowers of | flower color | flower color |
| surface | RHS 155B (White) | with a grey | with a grey |
| | | center | center |
| Plant growth habit | Semi-erect | Semi-erect | Compact |

‘SAKOST8202’ is a distinct variety of Osteospermum owing to its light pink colored flowers and a semi-erect plant habit. ‘SAKOST8202’ is most similar to the Osteospermum plant named ‘Arusha’. However, there are differences as described in Table 2. Color references are to The Royal Horticultural Society Colour Chart, 4^{th} Edition.

TABLE 2
Comparison with Similar Variety

| Characteristic | ‘SAKOST8202’ | ‘Arusha’ |
|---|---|---|
| Ray floret (ligule) color, | RHS 62C (Red-Purple) | Pink, Light Pink |
| upper surface: | with young flowers of RHS | and White |
| | 155B (White) | |
| Plant habit, Branching | Good | Poor |
| Plant habit, Vigor | Compact | Vigorous |
| Flower Size | Medium-large | Small-medium |

I claim:

1. A new and distinct variety of Osteospermum plant named ‘SAKOST8202’ as shown and described herein.
